(19) United States
(12) Patent Application Publication
(10) Pub. No.: US 2010/0152392 A1
(43) Pub. Date: Jun. 17, 2010

(54) CROSSLINKER FOR VULCANIZATION, PROCESS FOR ITS PREPARATION AND RUBBER PRODUCTS

(76) Inventors: Iourii Gribov, Berlin (DE); Pavel Kazimirskiy, Berlin (DE)

Correspondence Address: FURR LAW FIRM, 2622 DEBOLT ROAD, UTICA, OH, 43080, US

(21) Appl. No.: 12/447,903

(22) PCT Filed: Oct. 31, 2007
(86) PCT No.: PCT/EP07/61780

§ 371 Date: Apr. 29, 2009

(30) Foreign Application Priority Data
Oct. 31, 2006 (DE) 10 2006 052197.8

(51) Int. Cl. C08C 19/20; C07D 221/00

(52) U.S. Cl. 525/332.7; 546/242

(57) ABSTRACT

In order to provide a crosslinker for vulcanization, a process for its preparation and correspondingly produced rubber products, what is proposed is the reaction of nitrogen-containing heterocyclic compounds with sulphur at a temperature below 100° C. and the use of the resulting crosslinker for vulcanization.

The present invention relates to a crosslinker for vulcanization, to a process for its preparation as well as to suitably manufactured rubber products.

In addition to the vulcanization with elemental sulphur, which is known as hot vulcanization, there exists the possibility of crosslinking at low temperature. This is so called low-temperature vulcanization. Instead of sulphur, dissolved sulphurdichloride or disulphurdichloride is usually used.

Low-temperature vulcanization (T<100° C.) was previously used mainly in the manufacture of thin films. Rubber gloves were produced e.g. by immersion of a body-form into a latex emulsion, followed by immersion in a solution of S₂Cl₂ in CS₂, gasoline or benzene. This results in bridges, which include only one sulphur atom. The resulting hydrochloric acid is neutralized in an atmosphere of NH₃ and the excess of the disulphide dichloride is decomposed.

There are a number of known accelerators of the vulcanisation of rubber compounds based on caoutchouc, e.g. mercaptobenzothiazoles, sulphenamides, tetramethyltiuram.

Thus, the aim of the invention is to create a crosslinker for low temperature vulcanization, additionally the process of manufacturing such a crosslinker, and one which avoids the disadvantages of the prior state of technology.

The solution of the problem is a crosslinker, a method of its manufacture as well as of rubber products manufactured using the crosslinker, according to the claims 1, 15 and 16.

The crosslinker related to the invention is based on a mixture of nitrogen- containing heterocyclic compounds and sulphur, mostly in powder form.

The preferred heterocyclic compounds are here piperidine, pyridine, and anabasin and α,α′, β,′β,γ,γ′-dipyridile and particularly preferred is piperidine.

According to another embodiment of the invention, in addition to the aforementioned heterocyclic compounds another market standard crosslinker or accelerator with sulphur may be implemented.

These can be selected, for example, from the group of mercaptobenzothiazoles, sulphenamides, tetramethyltiuram or their derivatives.

The nitrogen-containing, heterocyclic connection reacts with sulphur at a temperature below 100° C., thus obtaining the desired crosslinker. Preferably, the reaction temperature of the mixture is kept below the boiling temperature of the heterocyclic connection.

Either the heterocyclic compound or sulphur can be submitted first for the reaction.

Due to the mild conditions possible, the reaction may be carried out under reduced pressure and/or cooling.

The temperature can be maintained still further after complete mixing of the two components, preferably by stirring. Preferably at 65° to 70° C. over 2 to 3 hours.

The mixing is done by a normal stirrer or mixer, such as a centrifugal mixer, propeller mixer, magnetic mixer, ultrasonic mixer and/or vibration mixer. A mixture may also be arrived at by use of turbulent flows.

The mixture can be made under inert gas.

The objects of this invention are the crosslinker, the method of its manufacture, and the rubber products, which are manufactured using the crosslinker.

Rubber products can also be produced in accordance with the details of the present invention using the crosslinker related to their invention in conjunction with other commercially available crosslinkers and/or accelerators.

The compounds used in accordance with the details of the present invention act as multifunctional agents in addition to their effect on the vulcanising reaction of the sulphur. Thus, they can act as plasticizers and stabilizers for fixation and/or modulation of vulcanization, i.e. for ensuring a defined grade of vulcanisation and for accelerating the vulcanisation processes. The amino structure serves to initiate the reaction of free radicals of sulphur.

The crosslinkers used in accordance with the details of the present invention, can be used both for vulcanisation at high temperatures as well as for low-temperature vulcanisation, whereby the low-temperature vulcanisation is particularly to be preferred. Any rubber mixtures can be used for vulcanization, such as, e.g. rubber compounds based on natural caoutchouc and synthetic mixtures, such as chloroprene, nitrile, Na-butadiene-styrene caoutchoucs and the like.

The method related to the invention is completely environmentally safe, where the entire raw material is implemented into the crosslinker.

The implementation runs very quickly and is economically advantageous, for no toxic auxiliary materials need to be used.

The process of vulcanisation with the crosslinker made in accordance with the details of the present invention has several advantages when compared to the methods used in the prior state of technology:
1) The crosslinker works as the vulcanization resource (vulcanization agent), and it is capable of vulcanising the rubber mixture in the absence of additional, usually very irregularly distributed sulphur powder, which increases the homogeneity of the network of the rubber and the heat stability of the resulting rubber products.
2) The crosslinker made in accordance with the details of the present invention serves also as the accelerator, because the process of vulcanisation occurs much faster. The resulting rubber compounds exhibit improved physical-mechanical data.
3) The crosslinker has the properties of primary plastiziers that increases the plasticity of the rubber compound.
4) The process of vulcanisation requires no addition of zinc oxide and the absence of zinc oxide will not retard the process.
5) The time for mixing of the crosslinker with the raw rubber compound is significantly reduced.
6) The crosslinker is distributed more evenly throughout the mass of the rubber compound also at low temperatures in a short time.
7) The crosslinker vulcanises rubber compounds at a temperature below 100° C. It is, however, also suitable in the range of temperatures usually used, when of course the reaction speed is correspondingly increased.
8) The crosslinker increases the plateau of vulcanization from the usual minutes up to hours.
9) The crosslinker improves the ozone resistance of rubber products.
10) The crosslinker made in accordance with the details of the present invention can lead to the vulcanisation of layers of arbitrary thicknesses, not possible under the prior state of technology.

The invention is explained below, using examples and figures.

It shows

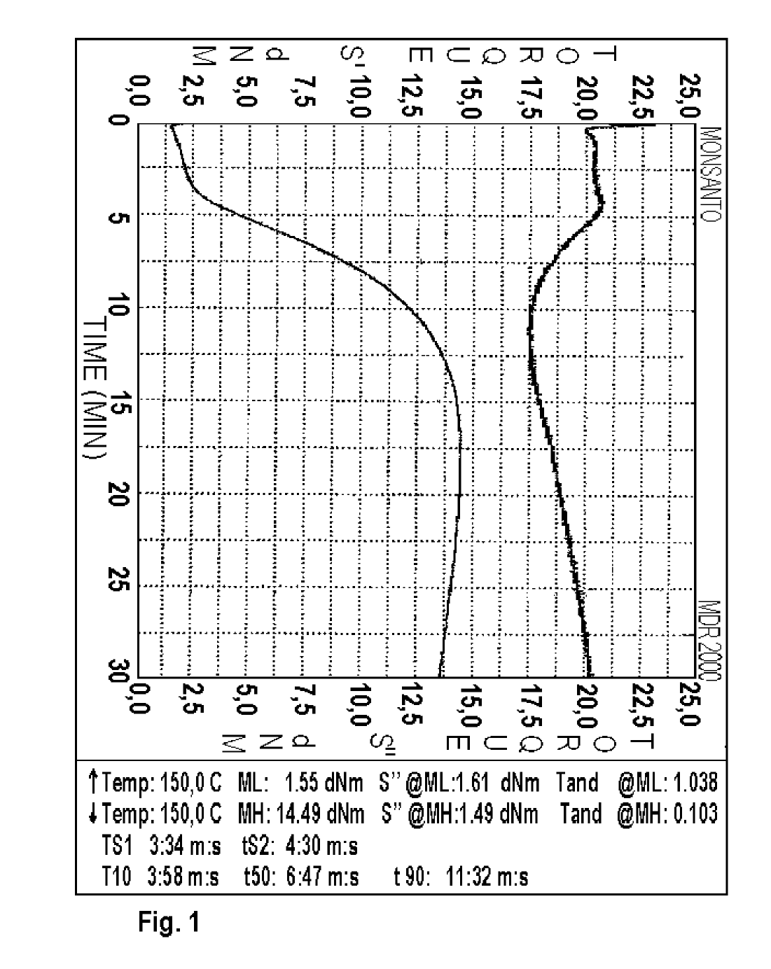

FIG. 1 Vulcanisation curve of a raw rubber compound, corresponding to the current state of technology, crosslinked with sulphur powder (Mixture 1),

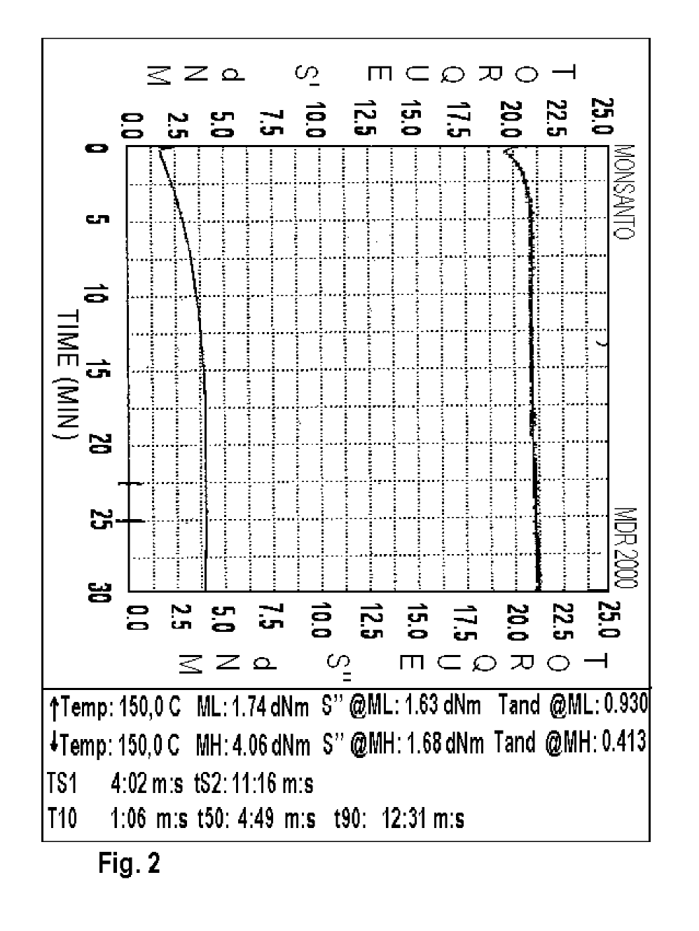

FIG. 2 Vulcanisation curve with the same raw rubber compound, crosslinked with the crosslinker made in accordance with the details of the present invention (Mixture 2),

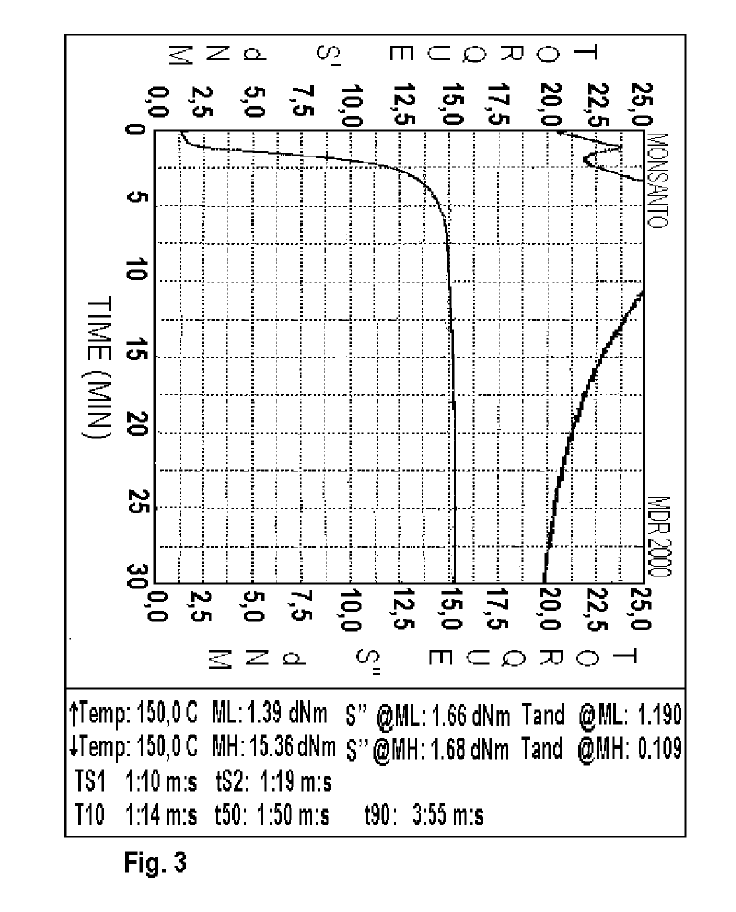

FIG. 3 Vulcanisation curve of the same raw rubber compound with another proportion of the crosslinker made in accordance with the details of the present invention (Mixture 3),

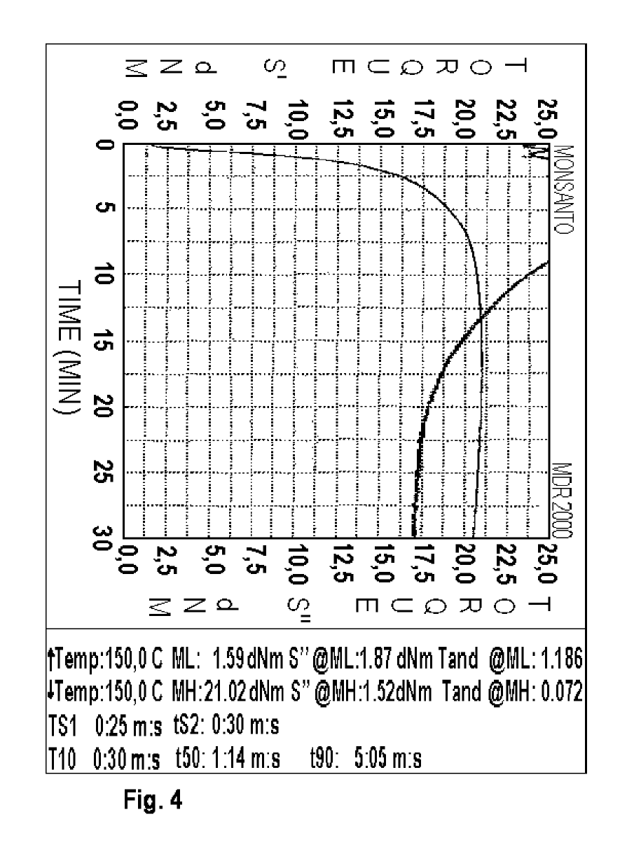

FIG. 4 Vulcanisation curve of the same raw rubber compound with another proportion of the crosslinker made in accordance with the details of the present invention (Mixture 4),

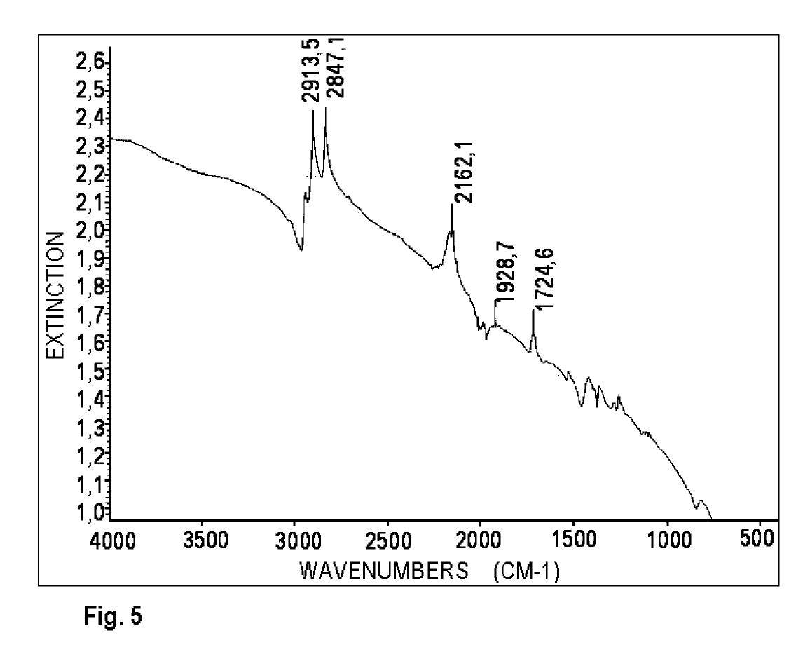

FIG. 5 IR spectrum of the same raw rubber compound, crosslinked according to the current state of technology with sulphur powder (Mixture 1),

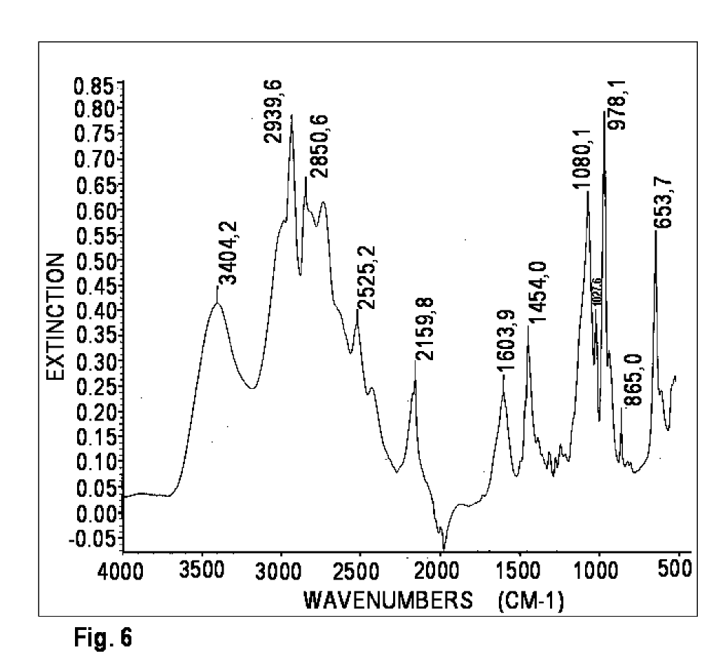

FIG. 6 IR spectrum of the crosslinker made in accordance with the details of the present invention,

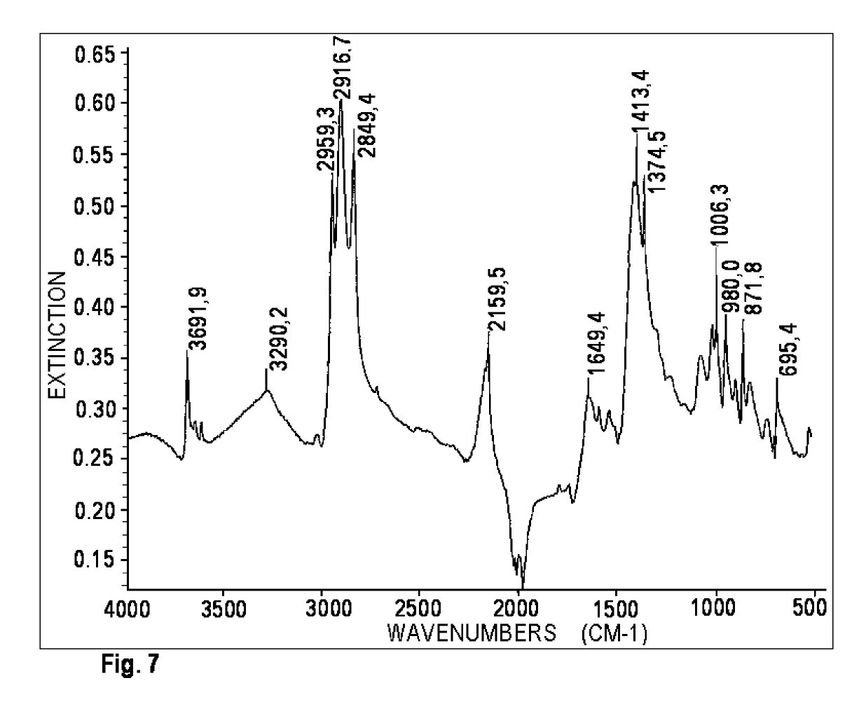

FIG. 7 IR spectrum of the same raw rubber compound, crosslinked by the crosslinker made in accordance with the details of the present invention (Mixture 3),

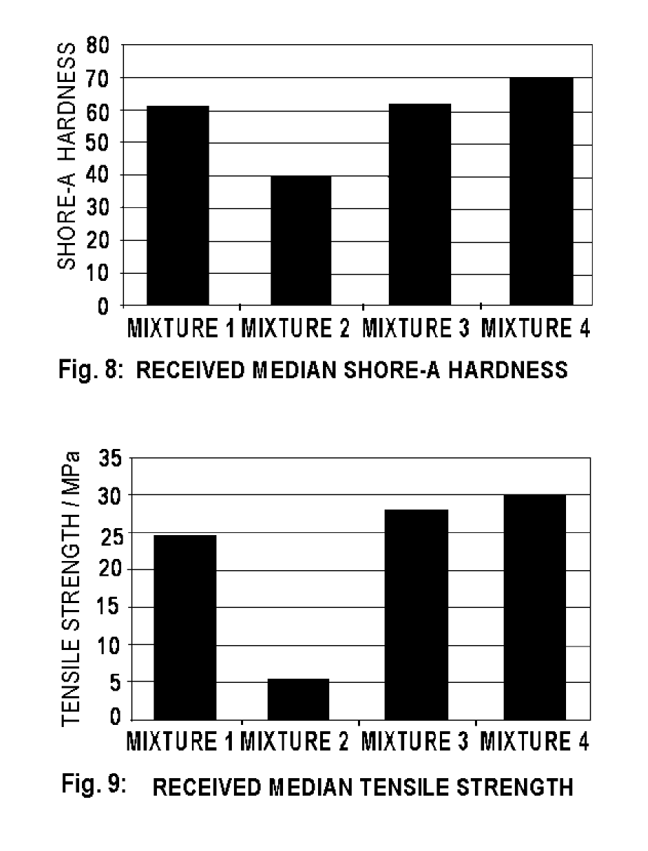

FIG. 8 Comparison of the Shore-A hardness,

FIG. 9 Comparison of the tensile strength and

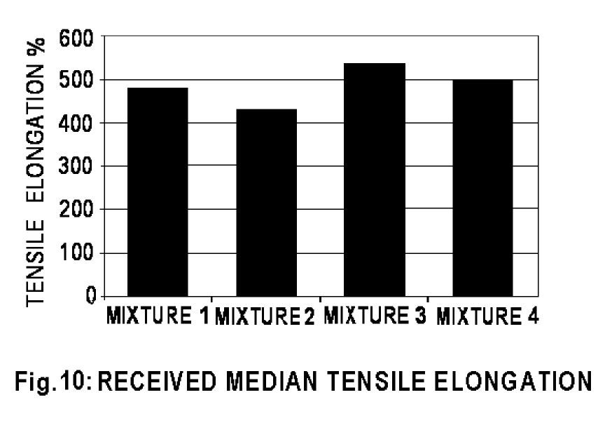

FIG. 10 Comparison of the tensile elongation.

### EXAMPLE 1

Synthesis of the Crosslinker Made in Accordance with the Details of the Present Invention

384 g of the powdered sulphur is placed in a flask, equipped with stirrer and thermometer. The flask is placed in a cooling bath of water. 396 ml of piperidine are added under stirring by the drip method. Alternatively, the piperidine is placed first and the sulphur powder is admixed in portions. The cooling of the reaction mixture is necessary, since the reaction is exothermic. After complete addition of the piperidine, the temperature of the reaction mixture is kept not higher than 65° to 70° C. The reaction mixture is recast into a plastic container after 2.5 h, and is cooled to 5° to 10° C. The cooled mixture of the reaction has the consistency of crystallized honey, and is a grey-brown-red colour. The melting point of the reaction product is about 60° C.

The synthesis can also be carried out as a continuous process, using turbulent mixing of the mixing materials, where this may also be done under an inert gas.

### EXAMPLE 2

IR-Spectroscopic Investigations

Device: Thermoelectron IR-spectrometer Type 380 with ART-Unity

The IR spectrum shows, according to FIG. 5, the relatively weak expression of the characteristic IR spectral lines and a relatively structureless crosslinking of the rubber manufactured according to the current state of technology (Mixture 1).

The reason for this is the poor mixing of the sulphur powder in the very viscous raw rubber compound which always occurs. On the other hand, the IR spectrum of the invention-related rubber (Mixture 3) shows, according to FIG. 7, clearly structured and expressed IR-spectral lines. The spectral bands of high intensity in the area of 1380 cm-1, 1413 cm-1 and 1460 cm-1 are caused by vibrations of the CH3, CS, CH2 groups. This shows up the increase of structural order and thus of a growing steady repetition of the relevant structural units of the polymer. Homogeneous formation has been achieved, the structural units of sulphur with the C═C-polymer group repeating consistently, as a result of the replacement of the sulphur powder by the liquid networking agent, which is quickly and homogeneously distributable within the raw rubber. The shift of the band at 1724 cm-1 (Mixture 1) to the 1649 cm-1 (Mixture 3) shows the strengthening of R—NH, C═C, C═O linkages in the new rubber.

The crosslinker from piperidine and sulphur, made in accordance with the details of the present invention, is characterized by the IR spectrum shown in FIG. 6. One can see, by comparing FIGS. 6 and 7, that the spectral bands of Mixture 3 and the crosslinker are partially congruent with each other, e.g. in the bands 1380 cm-1-1460 cm-1 , 1600-1650 cm-1 , 2850-2960 cm-1.

### EXAMPLE 3

Material-Technical Investigations

TABLE 1
Basic recipe of the manufactured elastomer-compounds, which has
also been used partly for the spectroscopic investigations.

| Notation | Mass proportions (phr) |
|---|---|
| Natural Rubber SMR-L | 100 |
| Zinc oxide | 5 |
| Soot N 330 | 50 |
| Anti-aging agent IPPD | 1 |
| Stearic acid | 2 |
| Accelerator CBS | 0.25 |

Mixture 1:

| | |
|---|---|
| Crosslinker: | Sulphur |
| Crosslinker concentration: | 1.5 phr |
| Vulcanisation time: | 20 minutes |

Mixture 2:

| | |
|---|---|
| Crosslinker: | the invention-related crosslinker |
| | from piperidine and sulphur |
| Crosslinker concentration: | 0.75 phr |
| Vulcanisation time: | 25 minutes |

Mixture 3:

| | |
|---|---|
| Crosslinker: | the invention-related crosslinker |
| | from piperidine and sulphur |
| Crosslinker concentration: | 1.5 phr |
| Vulcanisation time: | 25 minutes |

Mixture 4:

| | |
|---|---|
| Crosslinker: | invention as crosslinker |
| | from piperidine and sulphur |
| Crosslinker concentration: | 3 phr |
| Vulcanisation time: | 20 minutes |

Vulcameter Measurements

The vulcanisation measurement was carried out with a vulcameter. Here a defined quantity of un-vulcanised mixture is placed between two metal chambers under pressure and temperature. The vulcanization begins, whereby bridges within the molecule are formed and the mixture becomes increasingly networked. The lower part of the chamber moves by a few angle degrees back and forth, while the upper chamber has a force sensor, which measures the deflection and transfers it to a recorder. As the results arrive, an envelope curve, which reproduces the degree of crosslinking for a given time, as well as the time needed for the material to reach the desired network proportion.

The vulcanization was accomplished at 150° C. in a multi-platen press, where plates with dimensions of 2 mm×186 mm×186 mm were manufactured.

The vulcameter measurements taken are shown in the Table 2.

There are:

Mₕ—maximal torque

Tₛ—scorch-time (time until onset of vulcanization)

T₉₀—Time for reaching 90% of torque expansion between the lowest torque and the Mₕ

TABLE 2
Results of the vulcameter measurements

| | t_{S1} (min:sec) | t_{S2} (min:sec) | t₉₀ (min:sec) | Mₕ (dNm) |
|---|---|---|---|---|
| Mixture 1 | 03:34 | 04:30 | 11:32 | 14 |
| Mixture 2 | 04:02 | 11:16 | 12:31 | 4 |
| Mixture 3 | 01:10 | 01:19 | 03:55 | 15 |
| Mixture 4 | 00:25 | 00:30 | 05:05 | 21 |

The measurements show that the crosslinker made in accordance with the details of the present invention allowed us to achieve vulcanisation significantly faster and with a very stable plateau of the curve than is possible with commonly used sulphur.

Shore A Hardness According to DIN 53505:

| | |
|---|---|
| Probe body: | glass, diameter 36 mm |
| Number of specimen | 3 discs, covered with 3 layers |

The median values of the measured hardness are presented and summarized in FIG. 8 and in Table 3.

TABLE 3
Results of the hardness measurements

Shore A hardness

| Notation | Median | Mean deviation |
|---|---|---|
| Mixture 1 | 61 | 2 |
| Mixture 2 | 40 | 4 |
| Mixture 3 | 62 | 5 |
| Mixture 4 | 70 | 1 |

Tensile Strength and Elongation After DIN 53504:

| | |
|---|---|
| Probe body: | shoulder stab S2 |
| Number of specimens: | each has 7 shoulder stabs |

The median values of the measured tensile strength are presented and summarized in FIG. 9 and in Table 4.

TABLE 4
Result of tensile strength according to the tensile test

Tensile strength in Mpa

| Notation | Median | Mean deviation |
|---|---|---|
| Mixture 1 | 24.6 | 4.4 |
| Mixture 2 | 5.4 | 2.9 |
| Mixture 3 | 27.8 | 4.7 |
| Mixture 4 | 30.1 | 1.2 |

The median values of the measured tensile elongation are presented and summarized in FIG. 10 and Table 5.

TABLE 5
Results of the tensile Elongation according to the tensile test

Tensile elongation in %

| Notation | Median | Mean deviation |
|---|---|---|
| Mixture 1 | 480 | 63 |
| Mixture 2 | 432 | 136 |
| Mixture 3 | 538 | 91 |
| Mixture 4 | 496 | 42 |

The investigations of the hardness and tensile elongation point show in the comparable Mixtures 1 and 3 significant or very significant improvement in the material properties of the invention-related rubber products.

What is claimed is:

1. Process of production of a crosslinker for vulcanization, characterized in, that nitrogen-containing, heterocyclic compounds with sulphur or with a mixture of sulphur with at least one or more usual crosslinkers or accelerators may be replaced.

2. Method in accordance with claim 1, characterized in, that the further crosslinkers/accelerators from the following group are selected: mercaptobenzothiazoles, sulphenamides, tetramethyltiuram or their derivatives.

3. Method in accordance with claim 1 or 2, characterized in, that the implementation takes place at a temperature below 100° C.

4. The method in accordance with claims 1 to 3, characterized in, that the sulphur is used in powder form.

5. Method in accordance with claims 1 to 4, characterized in, that the heterocyclic compound is selected from the following group: piperidine, pyridine, anabasin and α,α′,β,′β,γ,γ′-dipyridile.

6. Method in accordance with claim 5, characterized in, that the heterocyclic connection is piperidine.

7. Method in accordance with one of claims 1 to 6, characterized in, that the temperature of the mixture during the mixing process is kept below the boiling temperature of the heterocyclic connection.

8. Method in accordance with one of claims 1 to 7, characterized in, that the sulphur is placed at the beginning and the heterocyclic connection is added thereafter, where the addition of the heterocyclic connection is preferably carried out dropwise, in steps or in any other continuous way.

9. Method in accordance with one of claims 1 to 7, characterized in, that the heterocyclic connection is placed at the beginning and the sulphur is added thereafter, where the addition of the sulphur is carried out preferably in steps or any other continuous way.

10. Method in accordance with one of claims 1 to 9, characterised in, that the process is elaborated as a Batch-method or as a continuous process.

11. Method in accordance with one of claims 1 to 10, characterised in, that it works under reduced or elevated pressure.

12. Method in accordance with one of claims 1 to 11, characterised in, that it works under cooling.

13. Method in accordance with claim 12, characterised in, that at atmospheric pressure after complete mixing of the two components, the temperature is kept at 65° to 70° C. over a period up to 3 hours.

14. Method in accordance with one of claims 1 to 13, characterised in, that for the mixing of sulphur and heterocyclic compounds known mixers such as centrifugal mixers, propeller mixers, magnetic mixers or ultrasound mixers, vibratory mixers, dispersing, turbulence mixers or combinations of the above mentioned mixers are used.

15. Crosslinker for vulcanization, characterised in, that the crosslinker is produced according to the procedure according to one of the claims 1 to 14.

16. Rubber product manufactured using a crosslinker in accordance with claim 15, that where at least one other known accelerator is used in addition.

17. Rubber product in accordance with the claim 16, characterised in, that the rubber product is produced of known natural and/or synthetic rubber
